(19) United States
(12) Patent Application Publication
(10) Pub. No.: US 2022/0287623 A1
(43) Pub. Date: Sep. 15, 2022

(54) Intra-Vaginal Ring with Pressure Sensor

(71) Applicant: LiGalli B.V., Den Haag (NL)

(72) Inventor: Wilhelmus Nicolaas Gerardus Maria De Laat, Den Haag (NL)

(21) Appl. No.: 17/636,285

(22) PCT Filed: Oct. 10, 2020
(86) PCT No.: PCT/EP2020/078474

§ 371 Date: Feb. 17, 2022

(30) Foreign Application Priority Data
Oct. 10, 2019 (EP) 19202485.9

(51) Int. Cl. A61B 5/00; A61B 5/03; A61B 5/07

(52) U.S. Cl. CPC A61B 5/4337; A61B 5/036; A61B 5/746; A61B 5/7455; A61B 5/07; A61B 5/4839; A61N 1/36007

(57) ABSTRACT

The present invention relates to a device for determining intra-abdominal pressure in a subject, comprising an intravaginal ring that is provided with a pressure sensor, means for registering the intra-abdominal pressure value measured by the pressure sensor and means for generating an alert signal when the intra-abdominal pressure exceeds a reference value. The present invention also relates to a method for determining intra-abdominal pressure in a subject, comprising the steps of: a) measuring the intra-abdominal pressure in the vagina by means of a device of the invention, b) comparing the intra-abdominal pressure value measured with a reference value; and c) in case the intra-abdominal pressure value exceeds the reference value transmitting an alert signal to the device that triggers a vibration element in the device or an external device, such as a smartphone or smart watch, to provide a warning to change the subjects behaviour in order to lower the intra-abdominal pressure value.

### FIELD OF THE INVENTION

The present invention relates to a device for the measurement of intra-abdominal pressure. The invention further provides a method for such measurement.

### BACKGROUND OF THE INVENTION

Measuring intra-abdominal pressure (abbreviated as IAP) has been performed for over 50 years. There are many publications about IAP measurements and the (causal) relations with pelvic floor pathologies and involuntary urine loss (i.e stress-incontinence), surgeries for prolapses of the vaginal wall and reasons for recurrences.

Currently, measurements are usually done by urodynamic testing, a method to test pressures inside the bladder, urethra and the rectum, both with catheters inside these organs, in a set-up that occurs in the doctors office, hospital or a laboratory where the bladder is provoked to contractions via filling with different liquid solutions and different temperatures.

Nothwithstanding the long experience and refinement of the method, it seems that urodynamic testing is still controversial, as well in indications and results as in reproducibility. The supine position of the patient with elevated legs, inserted catheters and the presence of a docter and/or nurse at testing is considered as not representative for the physiological circumstances.

The vagina has also been confirmed as a suitable location to reliably measure IAP. Coleman et al. (Development of a wireless intra-vaginal transducer for monitoring intra-abdominal pressure in women. Biomed Microdevices 2012; 14 (2): 347-355) describe a cylindrical elastomeric capsule for intravaginally measuring IAP. The device of Coleman was tested by various other groups. The device, used in these studies, had the design of a capsule equipped with sensors and electronics and was modeled after a tampon. It was tested wired and later wireless and during performance of several different daily activities. Clinical evaluation initially validated the transducer as comfortable and easily retained.

However, it appeared that the variability in pressure recordings varied clearly between identical patients in different sessions, between different patients in similar exercises, and between different activities (Shaw et al.; Intra-abdominal pressures during activity in women using an intra-vaginal pressure transducer. J Sports Sci: 2014; 32 (12) 1176-1185). These results also showed that no reliable wireless registration and transmission could be established with the capsule device. The strength of the signal and the insecure positioning of the transducer in the vagina have been published as the reasons (Niederauer et al.; Development of a novel intra-abdominal pressure transducer for large scale clinical studies. Biomed Microdevices: 19(4) 80).

These studies dealt with pelvic floor problems where IAP increases allegedly influence the occurrence, surgery result or postoperative recurrence.

Restriction of activities after pelvic surgery is probably useful in supporting recovery, but until now unproven. This is inter alia caused by lack of the possibility to monitor IAP continuously and under normal daily circumstances. Restriction of activities could even result in adverse outcomes, but this has also not been proven. The inventors considered that wireless testing under more physiological daily circumstances should result in more relevant data than testing in a doctors office or laboratory setting, while the patient is equipped with multiple sensors and accompanied by investigators on location.

It is therefore an object of the present invention to provide the means for testing intra-abdominal pressure under more physiological circumstances.

### SUMMARY OF THE INVENTION

The invention thus relates to a device for determining intra-abdominal pressure in a subject, comprising an intravaginal ring that is provided with a pressure sensor, means for registering the intra-abdominal pressure value measured by the pressure sensor and means for generating an alert signal when the intra-abdominal pressure exceeds a reference value.

In a further embodiment, the device further comprises means for transmitting the pressure value measured to an external device, in particular a computer outside the subjects body.

The device can further comprise means for receiving instructions from the computer.

The means for generating an alert signal can comprise a vibrating element integrated in the ring or means for wireless transmission of an alert signal to an external device, or a combination thereof. In this context the external device is for example a smartphone or a smart watch.

The means for generating an alert signal are suitably configured to receive instructions from a computer outside the subjects body and/or from the pressure sensor.

In a further embodiment the device further comprises drug delivery means.

The drug delivery means are suitably configured to deliver a drug in response to the generation of an alert signal when the intra-abdominal pressure exceeds a reference value.

In another embodiment, the drug delivery means are configured to deliver a drug on demand.

The device may further comprises means for generating an electrostimulus.

The device of the invention can suitably take the form of the intravaginal ring as described in WO2017060299. The intravaginal ring thus comprises:

a first rigid member having a first and second end;

a second rigid member having a third and fourth end;

a first flexible member coupled between the first and third ends;

a flexible part coupled between the second and fourth ends;

optionally a reservoir holding a drug to be delivered, an opening, and a pump for pumping said drug out of said opening,

wherein the first flexible member and/or flexible part are configured for allowing the device to be squeezed by bringing the second and fourth ends together thereby transforming a shape of the ring from an extended shape to a collapsed shape for allowing the device to be inserted into a vagina of a subject at or near the fornix posterior vaginae, said extended shape corresponding to a substantially oval or annular ring shape, and
wherein at least one of the first flexible member and the flexible part is at least partially elastic such that the device is pre-biased to assume the extended shape when no external force is being applied thereto.

The present invention furthermore relates to a method for determining intra-abdominal pressure in a subject, comprising the steps of:

a) measuring the intra-abdominal pressure in the vagina by means of a device as described herein,

b) comparing the intra-abdominal pressure value measured with a reference value; and

c) in case the intra-abdominal pressure value exceeds the reference value transmitting an alert signal to the device that triggers a vibration element in the device or an external device, such as a smartphone or smart watch, to provide a warning to change the subjects behaviours in order to lower the intra-abdominal pressure value.

In a further embodiment, an instruction is transmitted to the drug delivery means to start releasing a drug into the vagina.

### DETAILED DESCRIPTION

The function of the device is to register an elevated IAP. When such elevated IAP is measured the subject is warned to change their behavior to bring the IAP down again. This is suitably done by some kind of alert signal. The alert signal can simply be a notification on an external device such as a computer. The computer can be in the doctors office, the hospital or a laboratory when the device is used in diagnosis.

In a preferred embodiment, the alert signal is directly transmitted to the subject themselves. This way they can immediately take action by changing their behavior.

In one embodiment, the alert signal is generated by the ring itself. To this end a vibration element may for example be integrated in the ring. Upon registering an elevated IAP the ring starts to vibrate and the subject is immediately warned by this.

In another embodiment, the alert signal is displayed on an external device, such as a computer or a tablet or on a mobile device such as a smartphone or a smart watch. The device suitably transmits the alert signal by wireless communication directly to the external device.

In another embodiment, the IAP value measured by the sensor is transmitted to an external device, such as a computer, in which it is determined whether the IAP exceeds a reference value. If this is the case, the computer sends an alert signal to the vibration element in the ring or to a mobile device carried by the user.

Alternatively, the device can be programmed to recognize an IAP value that exceeds a reference value and to directly generate an alert signal, either by activating the vibration element or by sending the alert to the mobile device.

In the device of the present invention the pressure sensor is in firm and secure contact with the vaginal wall because the sensor is mounted on an intravaginal ring, in particular a ring with elastic properties. The flexible ring expands to the vaginal walls after insertion and reaches a stabilized position hereby ensuring a continuous contact of the sensor with the vaginal wall closest to the bladder or any other position of the ring. This enables a consistent pressure sensor registration. In contrast, the capsule device of the prior art has no firm and no fixed contact with the vaginal walls. When the musculature of the user between the vagina and the rectum is weakened the tampon-shaped device will move in the direction of the rectum in case of pressure elevation. During this movement the device encounters no resistance and as a result the IAP values measured can be incorrect. In the present device when using the ring as described in WO2017060299, this can not happen because the positioning of the rigid part (the hinge) in the fornix posterior prevents movement as a result of counterforces of the sacrouterine ligaments.

The ring shape of the device also allows use of a sufficiently long antenna. This feature makes it possible to wirelessly transmit registration data from the sensor to an external device for data analysis and transmitting alerts. This opens the window for ambulant measurements of patients under normal daily physiological circumstances to provide more reliable results.

The device can also be used in a method to guide patients with a prolapse surgical operation. Postoperatively, these patients are advised in many different ways how to behave to guarantee an optimal result of the operation. Today the advises differ wildly (avoid lifting, hoisting, sneezing, etc.), but are never based on objective, quantitative signals.

In one embodiment, an alert is generated when the intra-abdominal pressure reaches a value which is considered harmful for the healing of tissues after pelvic or abdominal surgery, in particular pelvic floor muscles and vaginal tissue.

The ring can be used for measuring additional parameters with one or more other sensors. It is for example possible to measure IAP as well as intravesical pressure at the same time either with only one sensor that measures both or with two or more sensors.

The device can further comprise drug delivery means to integrate diagnosis with therapy in a vaginal ring.

The drug delivery means can be used for delivering any medicament that has a function in treating IAP, abdominal surgery and related indications. The device can for example be used for estrogen delivery in postmenopausal women to improve the recovery process of the vaginal wall after surgery.

The device can be used as part of a so called uro-dynamic investigation (UDI) to diagnose overactive bladder (OAB) with signal analysis using vaginal pressure sensoring.

When the device is provided with drug delivery means delivery of a drug, such as anticholinergics and beta-adrenergics, in particular oxybutynin, can be triggered on-demand by a pressure sensor signal, or automated in a closed loop between diagnostic sensor signal and drug delivery means. A relation of intravaginal pressure changes with episodes of OAB in an ambulant setting is novel. Wireless intravaginal pressure sensors have not yet been used to record the IAP under normal daily ambulant circumstances and evaluate the relation with involuntary urine loss episodes.

Another aspect of the invention relates to the generation of an electrostimulus to muscles, for example pelvic floor muscles. The electrostimulus can be triggered on-demand by a pressure sensor signal, or automated in a closed loop between diagnostic sensor signal and the electrostimulus generating means. This allows for the generation of personalized, programmable electrostimuli to pelvic floor or other abdominal muscles.

The device of the invention can be used in a variety of other diagnostic methods of indications involving elevated IAP, optionally combined with treatment. It can for example be used to diagnose specific patterns of bowel diseases and assess improvement by treatment (e.g. irritable bowel disease, Crohn disease, chronic obstipation), or to assess IAP change at treatment of COPD. IAP is known to be elevated by lung obstruction. It is also possible to continuously register IAP for several weeks to assess longterm individual variations. The ring can also be used for replacement of an intrarectal pressure catheter as used in conventional urodynamic testing.

The invention thus enables ambulatory/out-of-clinic testing of intra-abdominal pressure changes under normal daily circumstances and activities, because the measurement results are wirelessly communicated with a remote computer/handheld device. The ring can therefore serve as a replacement of conventional urodynamic testing (in a clinical setting).

The invention facilitates post-operative recovery after surgery for prolapses and abdominal hernia's by restricting and avoiding too high intra-abdominal pressures via warning signals via handheld devices.

The device can furthermore include a gyroscope sensor to measure the IAP in relation to body posture.

One intravaginal ring that is particularly suitable for use in the device of the invention is described in WO2017060299.

The device of the invention can be used in a method for determining intra-abdominal pressure in a subject, wherein the intra-abdominal pressure is measured in the vagina by means of the device as herein described, comparing the intra-abdominal pressure value measured with a reference value; and in case the intra-abdominal pressure value exceeds the reference value transmitting an alert signal to the device that triggers a vibration element in the device or an external device, such as a smartphone or smart watch. In this way an immediate warning is given to change the subjects behaviour in order to lower the intra-abdominal pressure value.

The pressure sensor and other sensors can mounted on the ring but are suitably integrated in the ring structure.

In addition to measuring the IAP and generating a warning signal in case the IAP value exceeds a reference value an instruction can be transmitted to the drug delivery means to start releasing a drug into the vagina.

The present invention will be further illustrated in the example that follows and that is given for illustration purposes only and is not intended to limit the invention in any way.

### FIGURES

In the example reference is made to the following figures:

FIG. 1: photograph of an intravaginal ring having a sensor mounted thereon.

FIGS. 2 and 3: details of the sensor.

FIG. 4: Specification sheet of the Sentron Full Bridge Pressure Sensor P4.3 (E7500349.01). The P4.3 pressure sensor is an advanced miniature pie-zoresistive sensor equipped with a full Wheatstone bridge. The full Wheatstone bridge ensures superior linearity and temperature characteristics and Sentron's advanced processing technology permits the dimensions of the sensor (L×W×H 1770×970×300 m) to remain very small. The short sensor length allows a smaller bending radius and reduces stresses on the sensor during catheter bending.

FIG. 5: registration of IAP measured intravaginally with the device of the invention and through conventional intravesical measurement.

### EXAMPLE

In a pilot, a conventional UDI was performed with an intravaginal device of the invention (instead of the usual rectal sensor) and an intrabladder sensor. The UDI ran for 30 minutes. Intra-abdominal pressure was provoked by asking the patient to cough a couple of times. The bladder was slowly filled with water.

The IAP was recorded with the intravaginal pressure sensor mounted on the vaginal ring.

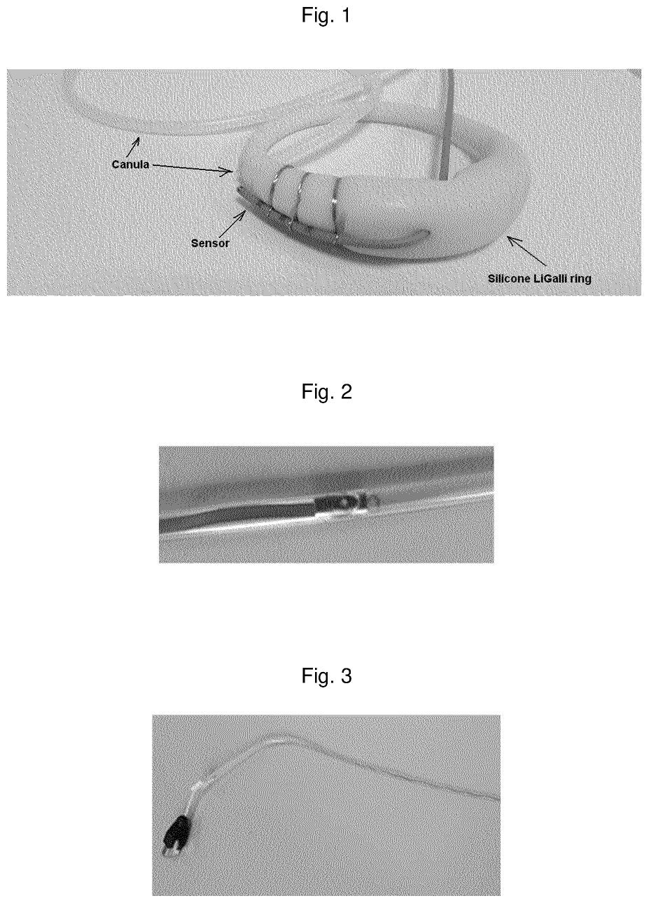

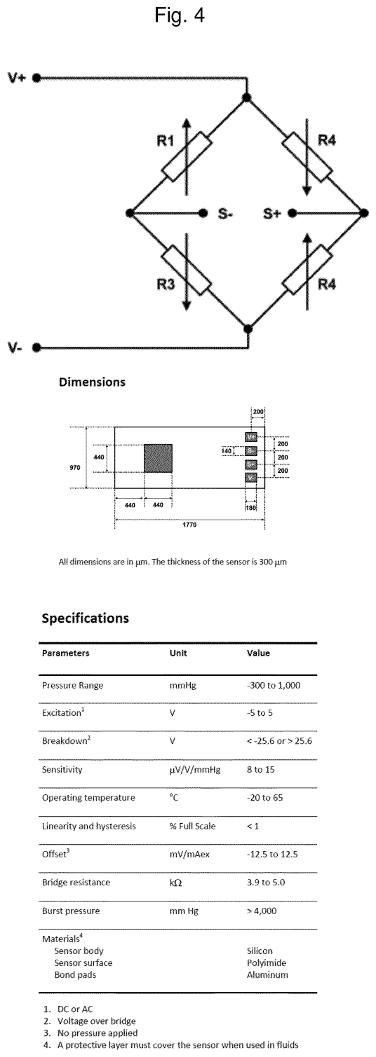

The pressure sensor used was provided by Welling company in Leek, the Netherlands (see FIGS. 1, 2 and 3) according to the attached specifications (FIG. 4)

The recording device used was an Uromic Jive (Medkonsult).

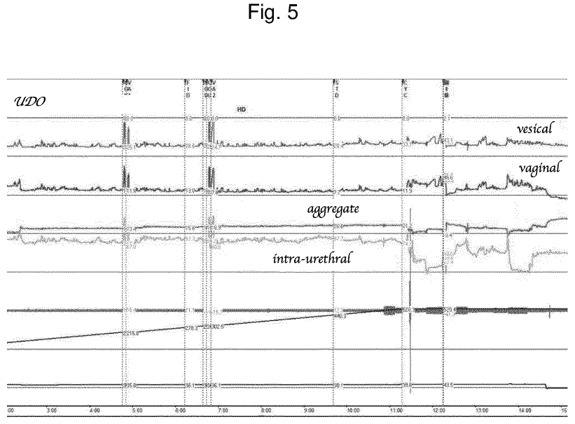

FIG. 5 shows that the intravaginal pressure sensor (middle line, labeled “vaginal”) clearly registered the intra-abdominal pressure registration when compared with the intravesical conventional measurement (upper line, labeled “vesical”). The vaginal pressure recording showed a signal closely resembling the intravesical pressure recording.

A vaginal ring mounted with a pressure sensor clearly registers intra-abdominal pressure differences. Because the device of the invention is suitably provided with transmission means, it can send the results online to a computer. This opens the possibility for mobile UDI's with longer registrations under more physiological circumstances than in the doctors office with various catheters and bladder provocations.

It also opens the possibility to guide patients through the difficult post-operative period, if the device can signal to the patient (for example via an app) when too much intra-abdominal pressure is applied during normal daily activities endangering the healing process.

1. Device for determining intra-abdominal pressure in a subject, comprising an intravaginal ring that is provided with a pressure sensor, means for registering the intra-abdominal pressure value measured by the pressure sensor and means for generating an alert signal when the intra-abdominal pressure exceeds a reference value.

2. Device as claimed in claim 1, further comprising means for transmitting the pressure value measured to an external device, in particular a computer outside the subjects body.

3. Device as claimed in claim 3, further comprising means for receiving instructions from the computer.

4. Device as claimed in claim 1, wherein the means for generating an alert signal comprise a vibration element integrated in the ring.

5. Device as claimed in claim 1, wherein the means for generating an alert signal comprise means for wireless transmission of an alert signal to an external device.

6. Device as claimed in claim 5, wherein the external device is a smartphone or a smart watch.

7. Device as claimed in claim 1, wherein the means for generating an alert signal are configured to receive instructions from a computer outside the subjects body.

8. Device as claimed in claim 1, wherein the means for generating an alert signal are configured to receive instructions from the pressure sensor.

9. Device as claimed in claim 1, further comprising drug delivery means.

10. Device as claimed in claim 9, wherein the drug delivery means are configured to deliver a drug in response to the generation of an alert signal when the intra-abdominal pressure exceeds a reference value.

11. Device as claimed in claim 9, wherein the drug delivery means are configured to deliver a drug on demand.

12. Device as claimed in claim 1, further comprising means for generating an electrostimulus.

13. Device as claimed in claim 1, wherein the intravaginal ring comprising:
a first rigid member having a first and second end;
a second rigid member having a third and fourth end;
a first flexible member coupled between the first and third ends;
a flexible part coupled between the second and fourth ends;
optionally a reservoir holding a drug to be delivered, an opening, and a pump for pumping said drug out of said opening,
wherein the first flexible member and/or flexible part are configured for allowing the device to be squeezed by bringing the second and fourth ends together thereby transforming a shape of the ring from an extended shape to a collapsed shape for allowing the device to be inserted into a vagina of a subject at or near the fornix posterior vaginae, said extended shape corresponding to a substantially oval or annular ring shape, and
wherein at least one of the first flexible member and the flexible part is at least partially elastic such that the device is pre-biased to assume the extended shape when no external force is being applied thereto.

14. Method for determining intra-abdominal pressure in a subject, comprising the steps of:
a) measuring the intra-abdominal pressure in the vagina by means of a device as claimed in claim 1,
b) comparing the intra-abdominal pressure value measured with a reference value; and
c) in case the intra-abdominal pressure value exceeds the reference value transmitting an alert signal to the device that triggers a vibration element in the device or an external device, such as a smartphone or smart watch, to provide a warning to change the subject's behavior in order to lower the intra-abdominal pressure value.

15. Method as claimed in claim 14, wherein an instruction is transmitted to the drug delivery means to start releasing a drug into the vagina.
